(19) United States
(12) Patent Application Publication
(10) Pub. No.: US 2010/0147298 A1
(43) Pub. Date: Jun. 17, 2010

(54) INFANT PACIFIER FLUID DELIVERY DEVICE AND PACIFIER ASSEMBLY

(75) Inventors: Thomas C. Loescher, Rancho Sante Fe, CA (US); Dennis L. Fitzwater, Strawberry, AZ (US)

Correspondence Address: KNOBBE MARTENS OLSON & BEAR LLP, 2040 MAIN STREET, FOURTEENTH FLOOR, IRVINE, CA, 92614, US

(73) Assignee: A PLUS MEDICAL, Carlsbad, CA (US)

(21) Appl. No.: 12/636,458

(22) Filed: Dec. 11, 2009

Related U.S. Application Data
(60) Provisional application No. 61/122,234, filed on Dec. 12, 2008.

(51) Int. Cl. A61M 15/08; A61J 17/00

(52) U.S. Cl. 128/203.22; 606/234; 606/236

(57) ABSTRACT

A fluid delivery device for being secured on an infant pacifier comprises a fluid delivery nozzle configured to direct fluid toward an infant's nares and a nozzle support member secured to the fluid delivery nozzle and configured for being secured on the mouth shield of the pacifier.

### CROSS REFERENCE TO RELATED APPLICATIONS

This application claims priority to U.S. Provisional Patent Application No. 61/122,234 filed Dec. 12, 2008 and is incorporated herein by reference.

### BACKGROUND OF THE INVENTION

Infant pacifiers have been used for many years to provide sucking action for infants, calming them, assisting jaw development, and reportedly reducing the risk of SIDS. The American Academy of Pediatrics recommends use of the infant pacifiers up to about age one. A typical pacifier comprises a plastic mouth shield plate with a latex or silicone nipple mounted on the proximal side, and on the opposite distal surface is a handle or knob on which a handle ring may be attached. Such pacifiers, their commercial availability, disposability features and advantages are well known to parents, nurses, and others who administer care for infants and babies.

Because an infant pacifier is positioned near the infant's nose, and because most new born infants breathe exclusively through their nose for at least a few months, various pacifier designs and modifications for administering respiratory care to infants via a pacifier have been proposed. Examples of such pacifiers are described in U.S. Pat. Nos. 5,904,140, 5,868,131, 7,318,433, 6,776,157, 6,557,548 and 7,134,432. However, the medical pacifiers described in the aforesaid patents require special tooling designs, manufacturing equipment and assembly, and do not use the readily commercially available, inexpensive and disposable pacifiers to which parents have been accustomed for many years. The patented pacifiers are thus relatively expensive and are not readily available in convenient retail drugstores or grocery stores where typically a variety of different brands of the well-known infant pacifiers are readily available, allowing the parent to select the brand which they feel is most desirable and/or to which they are accustomed.

### SUMMARY OF THE INVENTION

The invention described herein is directed to a fluid delivery device for being secured on a readily commercially available infant pacifier for directing an aerosol or oxygen or an oxygen-containing gas to an infant using the pacifier. The device includes a nozzle support member for being secured on the mouth shield of the pacifier and a fluid delivery nozzle secured on the nozzle support member for directing fluid toward the infant's nares. The nozzle support member comprises a flexible material for being secured on the front or distal surface of the pacifier mouth shield, which surface faces away from the infant. In one preferred embodiment, the nozzle support member is provided with an adhesive surface, preferably a contact adhesive, which can be readily and efficiently secured on the distal mouth shield surface by simply pressing the adhesive-coated nozzle support member against the mouth shield surface.

The fluid delivery nozzle may be separate and attached to the nozzle support member during assembly or by the user. In a preferred embodiment, the fluid delivery device is a unitary structure with the nozzle support member and fluid delivery nozzle made from the same flexible material as a single molded or otherwise formed unitary structure.

A major advantage of the fluid delivery device described herein is its adaptability for being easily and efficiently secured on commercially available infant pacifiers. Such an advantage allows a parent or other attendant for the infant to select, obtain, and/or use substantially any presently commercially available infant pacifier, and when desired, simply mount the fluid delivery device on the infant pacifier mouth shield. The resulting infant pacifier assembly with mounted fluid delivery device may be used and reused as needed or desired, and thereafter, the fluid delivery device removed from the pacifier and discarded. Other advantages, features, designs and preferred embodiments as well as use of the fluid delivery device for delivering aerosol or oxygen to an infant using the pacifier will be described further hereinafter.

### BRIEF DESCRIPTION OF THE DRAWINGS

FIG. 1 is a front, distal perspective view of an infant pacifier with the fluid delivery device mounted thereon;

FIGS. 2 and 3 are side and front views, respectively, of the assembly of FIG. 1;

FIG. 4 is a plan view of the inside, proximal mouth shield surface of a pacifier showing a mounted nipple and a fluid delivery nozzle positioned at the upper edge of the shield;

FIGS. 5-7 are front perspective, side and top views, respectively, of a pacifier assembly showing an alternative nozzle support member embodiment;

FIG. 8 is a perspective view illustrating an alternative fluid delivery nozzle embodiment; and

FIG. 9 is a side view of a pacifier assembly including a fluid delivery device and an aerosol tube with one end secured on the fluid delivery nozzle and the opposite end secured on a nebulizer, and showing another nozzle support member embodiment.

### DETAILED DESCRIPTION OF PREFERRED EMBODIMENTS

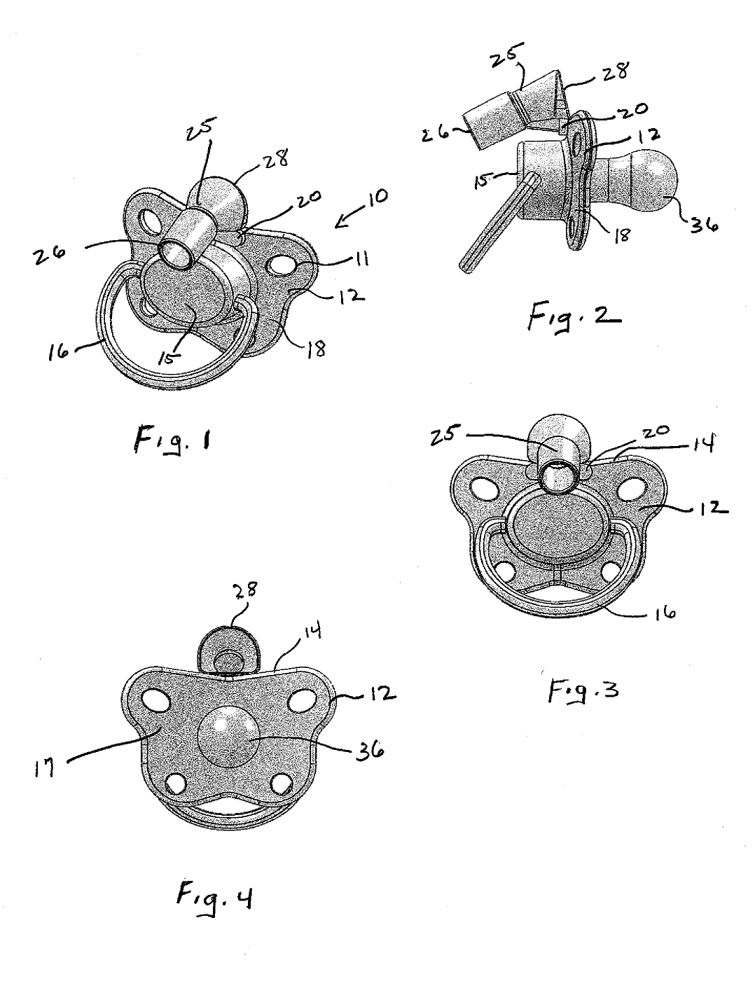

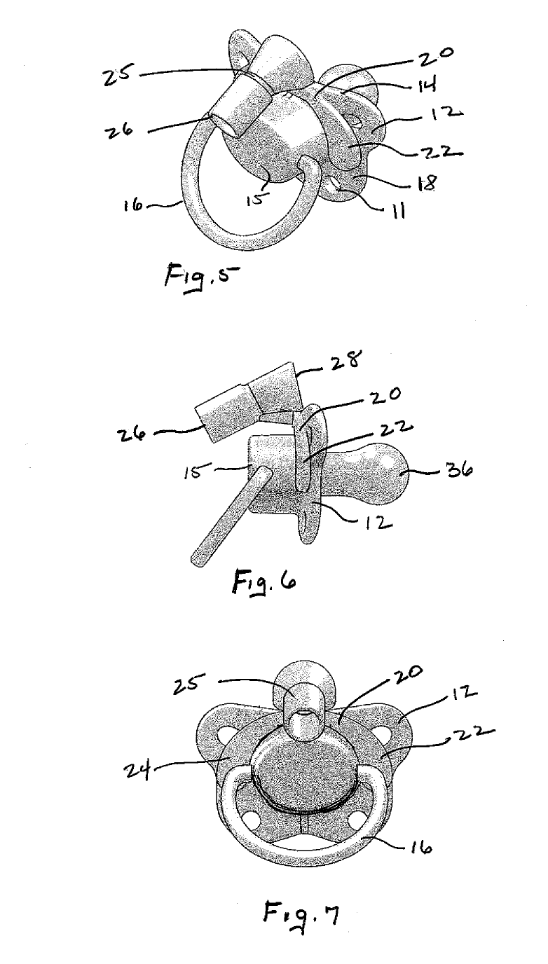

Referring to FIGS. 1-4, there is shown the infant pacifier assembly 10 which includes the infant pacifier on which is mounted the fluid delivery device of the invention. The pacifier may be any commercially available pacifier having a mouth shield 12, a handle 15, typically in the shape of a knob, as shown, and a nipple 36. The mouth shields of the different commercially available infant pacifiers have somewhat different shapes, with primarily different perimeter and handle shapes or configurations, but all include two or more ventilation holes 11 extending through the mouth shield to allow an infant to breathe in an unlikely event that the mouth shield is sucked into the infant's mouth. The handle 15 extends outwardly from the outward facing or distal surface 18 of the mouth shield. The handle may include a handle ring 16 which typically pivots or swivels on the knob. The nipple, mounted on and extending from the infant facing proximal surface of the mouth shield, is a flexible material, often made of latex or silicone.

The fluid delivery device of the invention is secured on the mouth shield of the infant pacifier. The fluid delivery device comprises a nozzle support member 20 and a fluid delivery nozzle 25. The fluid delivery nozzle and nozzle support member may be separate components, independently formed and assembled by the end user, or by the manufacturer. The two components may be joined by various means such as stapling the components together, or providing pins, posts or other protuberances extending from one of the components for being inserted into mounting orifices in the other component. Such a feature is shown in FIG. 10, discussed hereinafter.

In one preferred embodiment, the nozzle support member and fluid delivery nozzle are a unitary structure, formed of a common material and molded together as a single structure.

As shown in FIGS. 1-3, the nozzle support member is preferably generally symmetrical and comprises a flange 20 secured to and extending from the nozzle 25. The specific shape and size of the support member flange is not critical so long as it has adequate surface for being mounted onto and against the mouth shield and adequately supports the fluid delivery nozzle. In the embodiment illustrated, the nozzle support member flange comprises a bib or tab extending downwardly from the fluid delivery nozzle 25.

In another preferred embodiment, the inside surface of the nozzle support member is provided with adhesive, preferably a contact adhesive, for easily mounting and securing the nozzle support member on the outside distal mouth shield surface. For example, the inside surface of the mounting support member flange may be coated with a contact adhesive, and a protective non-adhesive film or other protective material secured over the contact adhesive. To mount the nozzle support member, the user simply separates the protective film from the contact adhesive, aligns the nozzle support member for being properly mounted, and presses the exposed contact adhesive coated nozzle support member against the mouth shield surface, thereby securing the components together.

As an alternative, the nozzle support member may be mounted on the pacifier mouth shield using clips or other equivalent components or means (not shown) for engaging both the side and/or upper edge of the mouth shield and the nozzle support member flanges for mounting and securing the assembly components.

The fluid delivery nozzle 25 has a fluid inlet port 26 and a fluid outlet port 28. The fluid delivery nozzle is preferably generally funnel shaped with the fluid inlet port configured for receiving, mounting or otherwise attaching a fluid supply tube such as an aerosol delivery tube or pipe or a gas delivery tube. Accordingly, the fluid inlet port 26 is preferably annular for receiving and attaching such tubing or pipe. The fluid outlet port 28 is preferably larger in size than the dimension of the fluid inlet port and is configured for directing a fluid toward the nares of an infant. Thus, the preferred overall shape of the fluid delivery nozzle is tapered from a smaller inlet port to an enlarged outlet port. The edge of the fluid outlet port may be slanted or tilted and preferably does not extend substantially, if at all, beyond the inner proximal surface of the mouth shield, thereby keeping it separated or spaced from the infant. It is also shown that the fluid delivery nozzle is generally centrally positioned on the nozzle support member, at its approximate center, so that the overall shape, dimension and design of the fluid delivery device is symmetrical, for being symmetrically mounted on the symmetrical mouth shield with the fluid delivery nozzle exposed at the upper end of the fluid delivery device and with the fluid outlet port exposed over the upper edge 14 of the mouth shield thereby providing full delivery of the aerosol or gas from the nozzle to the infant.

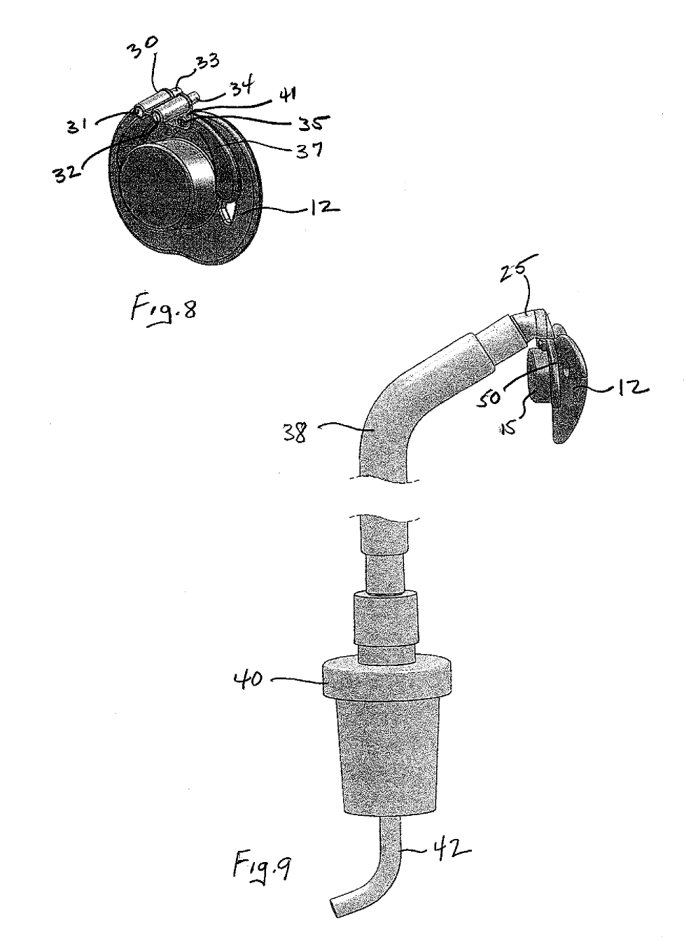

FIGS. 5-7 illustrate another embodiment of a fluid delivery device in which the nozzle support member comprises a pair of flanges or arms 22, 24 mounted on the distal surface 18 of the mouth shield 12. In this embodiment, the laterally extending arms are symmetrical and extend at equal distances from the nozzle that is mounted at the center of the support member. The specific dimensions and shape of the flanges is not critical so long as when mounted on the mouth shield, the fluid nozzle is adequately supported, and the arms do not substantially cover the ventilation holes. The inner surface of the nozzle support member including its arms or flanges may be provided with a contact adhesive as previously described, or such adhesive may be applied by the user prior to mounting and securing the device on the infant pacifier.

FIG. 8 illustrates another embodiment of the device of the invention utilizing an alternative means of securing or attaching a fluid supply nozzle on the nozzle support member. In the embodiment illustrated, the nozzle support member 37 includes a pair of mounting tabs or posts 35 extending through mounting holes formed on nozzle support flange 41 to support the fluid supply nozzle 30. In the illustrated embodiment, the fluid supply nozzle is configured with a pair of fluid nozzle inlets 31, 32 and a pair of fluid nozzle outlets 33, 34. This nozzle configuration may be used where more accurate or precise direction of the gas or aerosol delivery toward or into the nares of the infant via nozzle outlets 33, 34 is desired. The length of the fluid supply outlets may be designed to meet different patient's needs, as will be understood by those skilled in the art. The means of mounting and securing the fluid supply nozzle 30 on the nozzle support member 37 is only one example of using different alternatives for securing the components together, where they are not formed in a unitary body or structure as previously described. Other equivalent alternatives such as stapling, riveting, or gluing the fluid supply nozzle and nozzle support components together may be used.

FIG. 9 illustrates yet another embodiment of a fluid delivery device mounted on an infant pacifier. In this embodiment, the nozzle support member 50 is in the shape of a ring, donut or similar annular shape secured on the pacifier mouth shield around the knob 15 which extends outwardly from the mouth shield 12. Such a nozzle support member may be provided with adhesive on the inner ring surface for being adhered to the mouth shield or to the outer surface of knob 15, in a manner previously described regarding the contact adhesive in the embodiment illustrated in FIGS. 1-4. Alternatively, the nozzle support member 50 may be a resilient, elastic material stretched over and gripping the knob, thereby urging and holding the support member against the knob sufficiently to support the gas delivery nozzle adequately during its use. The fluid supply nozzle 25 illustrated is as previously described. The means for securing the fluid supply nozzle to the nozzle support member illustrated are like those shown regarding and described in FIG. 8, which is advantageous where the materials of the fluids delivery nozzle and support member are different. For example, the nozzle support member ring may be silicone or other resilient, elastic material, while the gas delivery nozzle may be made from a different material, e.g., polypropylene, polyurethane, etc. However, and alternatively, it may be desired to manufacture the fluid delivery device out of a single material such as a resilient and/or flexible plastic, such as silicone, or other similar synthetic resin material. In that case, the device can be molded as a single unitary structure as previously described.

FIG. 9 further illustrates the assembly with one end of a fluid supply tubing 38 directing an aerosol to fluid delivery nozzle 25 and the opposite distal end secured to a nebulizer 40 to which gas is supplied via gas tubing 42. Any suitable nebulizer may be used, although preferably a low volume nebulizer will be provided. Such a nebulizer, its operation and components are well known to those skilled in the art, and need not be further described herein. Alternatively, the fluid supply tubing secured to the fluid delivery nozzle may be secured to a oxygen source for delivering oxygen or oxygen-enriched gas to an infant via the device described hereinabove. The tubing may also include suitable fittings for convenient connection to the fluid nozzle inlet, or a simple force fit of the tubing to the nozzle inlet may be sufficient. The specific length of tubing secured to the fluid delivery nozzle will be sufficient to allow flexibility of the apparatus to be used with any desirable and remote aerosol-producing nebulizer or oxygen delivery source.

The fluid delivery device described herein may be molded out of relatively inexpensive plastic materials and may be easily and efficiently secured to as well as removed from commercially available and widely accepted infant pacifiers, without requiring substantial manufacture and assembly of pacifier devices which incorporate such fluid delivery components. Moreover, the fluid delivery devices described herein may be disposable along with, or independently of, the infant pacifier on which they are mounted, giving a parent or infant attendant significant flexibility in their pacifier selection as well as the use as described herein. The relatively inexpensive, reusable, disposable and easily mounted fluid delivery device described herein may be used for treating infant respiratory ailments by delivering oxygen or oxygen-enriched gas to an infant when indicated to improve blood oxygen levels or to maintain adequate tissue oxygenation where needed for documented or suspected acute hypoxemia. Similarly, the assembly should be easily adapted to deliver aerosol for treating infant asthma or for other respiratory care. These as well as other advantages and uses will be understood by those skilled in the art.

What is claimed is:

1. A fluid delivery device for being secured on an infant pacifier comprising a pacifier mouth shield having an upper edge underlying an infant's nares and a nipple extending from a proximal surface of said mouth shield,
said fluid delivery device comprising:
a nozzle support member configured for being mounted on said pacifier mouth shield, and
a fluid delivery nozzle secured on said nozzle support member for directing fluid therefrom toward an infant's nares.

2. A fluid delivery device of claim 1 wherein said nozzle support member includes an adhesive surface for being secured on said pacifier mouth shield.

3. A fluid delivery device of claim 1 wherein said nozzle support member comprises clips configured to engage an edge of said pacifier mouth shield for mounting said nozzle support member thereon.

4. A fluid delivery device of claim 1 wherein said fluid delivery nozzle comprises a first port configured for attaching an aerosol or gas supply tube thereto and a second port for dispensing aerosol or gas therefrom.

5. A fluid delivery device of claim 1 wherein said fluid delivery nozzle is detachably secured to said nozzle support member.

6. A fluid delivery device of claim 1 wherein said fluid delivery nozzle and said nozzle support member comprise a unitary component.

7. A fluid delivery device of claim 1 wherein said nozzle support member comprises a single flange or tube extending from and secured to said fluid delivery nozzle.

8. A fluid delivery device of claim 1 wherein said nozzle support member includes a contact adhesive surface for being secured on said pacifier mouth shield.

9. A fluid delivery device of claim 1 wherein said nozzle support member comprises flanges extending laterally from opposite sides of said fluid delivery nozzle.

10. A fluid delivery device of claim 1 wherein said fluid delivery nozzle comprises a funnel having a smaller inlet port and a larger outlet port.

11. A fluid delivery device of claim 1 wherein said nozzle support member comprises a collar.

12. A fluid delivery device of claim 11 wherein said collar includes an adhesive surface for being secured on said pacifier mouth shield.

13. A fluid delivery device of claim 11 wherein said collar comprises a resilient composition and configured for being detachably secured on said pacifier mouth shield.

14. An infant pacifier assembly comprising:
a mouth shield having a proximal surface for contacting an infant's mouth and a nipple extending therefrom, and an opposite distal surface having a handle thereon; and
a fluid delivery device comprising:
a nozzle support member secured on said mouth shield, and
an elongated fluid delivery nozzle secured on said nozzle support member having a fluid outlet at one end thereof positioned above an upper edge of said mouth shield.

15. An infant pacifier assembly of claim 14 wherein said nozzle support member comprises one or more flanges having an inner surface with contact adhesive thereon for securing said flange on said mouth shield.

16. An infant pacifier assembly of claim 14 including an elongated fluid inlet tube having a proximal end secured on said fluid supply nozzle at said fluid inlet port and a distal end comprising an adapter configured for securing a nebulizer thereon.

17. An infant pacifier of claim 14 wherein said fluid outlet comprises a pair of outlet tubes configured to direct a fluid to a user's nares.

18. An infant pacifier assembly of claim 14 wherein said fluid supply nozzle is detachably secured on said nozzle support member.

19. An infant pacifier assembly of claim 14 wherein said mouth shield includes a handle extending from the distal surface thereof, and wherein said nozzle support member comprises a collar extending around said handle.

20. An infant pacifier assembly of claim 19 wherein said handle comprises a knob, and wherein said collar comprises a resilient material stretched around said knob and is secured thereon.
